(19) United States
(12) Patent Application Publication
(10) Pub. No.: US 2024/0221943 A1
(43) Pub. Date: Jul. 4, 2024

(54) COMPUTER IMPLEMENTED METHOD FOR SELECTING FUNCTIONAL BIOMARKERS TO IDENTIFY A TARGET CONDITION IN A SUBJECT

(71) Applicant: NEWMANBRAIN, S.L., Elche (ES)

(72) Inventors: Joaquin IBAÑEZ BALLESTEROS, Alicante (ES); Sergio MOLINA RODRIGUEZ, Torrevieja (ES); Carlos BELMONTE MARTINEZ, San Juan de Alicante (ES)

(73) Assignee: NEWMANBRAIN, S.L., ELCHE (ES)

(21) Appl. No.: 18/396,194

(22) Filed: Dec. 26, 2023

(30) Foreign Application Priority Data
Dec. 28, 2022 (EP) 22383301.3

(51) Int. Cl. G16H 50/20; G16H 10/60

(52) U.S. Cl. CPC G16H 50/20; G16H 10/60

(57) ABSTRACT

Computer implemented method for selecting functional biomarkers to identify a target condition in a subject, the method comprising the steps of: obtaining groups of equivalent physiological signals generated during a stimulation, comprising a first group of physiological signals from subjects presenting the target condition, and a second group of physiological signals from subjects of control condition, not presenting the target condition; identifying at least one frequency sub-band for each group of physiological signals; obtaining per of the at least one identified frequency sub-band at least a corresponding pattern; and selecting the one or more patterns having similarity values to the physiological signals of each group that differentiate the groups of physiological signals as functional biomarkers. The functional biomarkers may be used for training a classifier for identifying the target condition in a subject.

### CROSS-REFERENCE TO THE RELATED APPLICATION(S)

This application claims priority under 35 USC § 119 to European Patent Application No. 22383301.3 filed on Dec. 28, 2022, the contents of which are herein incorporated by reference in their entireties.

### BACKGROUND

### 1. Field

The disclosure relates to a computer implemented method for selecting functional biomarkers to identify a target condition in a subject, such as attention-deficit/hyperactivity disorder (ADHD). The disclosure also relates to a classifier for identifying the target condition using the selected biomarkers and to a computer implemented method for identifying the target condition using the trained classifier.

### 2. Description of the Related Art

Attention-deficit/hyperactivity disorder (ADHD) is recognized as a highly prevalent neurodevelopmental disorder in school-age children worldwide, often persisting into adolescence and adulthood, and frequently overlapped with other psychiatric comorbidities. It is currently accepted that ADHD is a complex, heterogeneous disorder, in which different expressions of impairment along with variable trajectories must be recognized in order to adopt personalized approaches that best target an individual. This is important because, even despite serious distress/impairments, many patients lead rewarding and productive lives when properly managed.

Diagnosis of ADHD is event today based mainly on clinical signs and symptoms that require a detailed evaluation by an expert clinician through interviews with parents/caregivers and/or the patient himself, if applicable. Noteworthy, diagnosis cannot be solely based on rating scales, neuropsychological test or brain imaging. Despite the criticisms that argue a risk of subjectivity, the current consensus supports the validity of the diagnostic criteria applied by well-trained professionals. However, even for a specialist, clinical evaluation is quite time-consuming and requires several visits to be thoroughly performed. Besides, the significant shortage of trained professionals also contributes to a frequent delay in diagnosis or even to overlook some cases. From a developmental perspective, an early diagnosis is very likely to be of value for more effective pharmacological and psychosocial interventions. In this view, there is a need for objective biomarkers as useful adjunctive indicators to alleviate the workload of diagnoses and treatment follow-up.

Numerous studies have tried to assess ADHD through different objective diagnostic tools, most using functional or structural MRI and EEG, with other modalities (MEG, EKG, etc.) being deployed less frequently, and with an increasing use of artificial intelligence (AI) techniques.

Noticeable efforts in MRI and fMRI were made under the initiatives of the “ADHD-200 Consortium”. Despite significant advances in understanding abnormalities related to brain maturation and function, neuroimaging findings in ADHD research cannot yet be used to support clinical practice due to a variety of concerns.

An alternative tool to assess ADHD worth to explore is functional near-infrared spectroscopy (fNIRS), which is characterized by being noninvasive, wearable, cost-effective, and deployable in more friendly/ecological settings. fNIRS has shown its usefulness in monitoring functional hemodynamic changes associated with cortical brain activation. Compared to other neuroimaging modalities, few fNIRS studies have been conducted to differentiate children with ADHD from healthy controls, some of them trying to improve classification by combining different modalities (e.g. EEG+fNIRS). Even fewer studies focused on single unimodal approaches by using “exclusively” NIRS data. For example, Monden et al (2015, “Individual classification of ADHD children by right prefrontal hemodynamic responses during a go/no-go task as assessed by fNIRS”) reported a classification accuracy of 85% with a sensitivity of 90% by analyzing ROC curves obtained from right prefrontal oxy-Hb activation data during a go/no-go task.

Using prefrontal cortex activation measures during an N-back task, Crippa et al. (2017, “The utility of a computerized algorithm based on a multi-domain profile of measures for the diagnosis of attention deficit/hyperactivity disorder”) achieved mean accuracies of 78% with 72% sensitivity and 82% specificity when a support vector machine (SVM) classifier was trained on data from deoxy-Hb. Also employing an N-back task and an SVM, Gu et al. (2018, “Identifying ADHD children using hemodynamic responses during a working memory task measured by functional near-infrared spectroscopy”) reached 86% of accuracy with oxy-Hb data measured in the prefrontal and temporal cortex]. It is worth noting that no correction for components of non-cerebral origin was applied to the fNIRS signals in the aforementioned studies, which is especially important when scanning the prefrontal cortex through the forehead, since functional extra- and cerebral responses are interrelated processes that overlap in fNIRS recordings and with a greater confounding effect for oxy-Hb.

Notwithstanding this known drawback of fNIRS, classification algorithms may achieve appreciable performance by learning some type of feature representation from the uncorrected NIRS data, but uncertainty about the nature and origin of the features hampers the interpretability of predictive models. In these studies, the features were based on some kind of measurement from the averaged fNIRS data across trials/epochs, a classic approach that, while often providing robust results, fails to uncover finer distinctive patterns embedded in the data.

It is also known that a rhythmic mental arithmetic task successfully induced cyclical hemodynamic fluctuations coupled to the task frequency (33 mHz), and that the oscillatory patterns were consistent across individuals both in superficial and deep fNIRS signals recorded in the fronto polar region. Spectral analysis also showed oscillatory activity at lower frequencies (<33 mHz) seen at rest and during mental task, and with a prominent peak around 5-10 mHz. Resting-state fMRI studies have reported that ADHD patients show significant differences in the low-frequency oscillations (LFO; 10-80 mHz) band across multiple brain regions, with separable contribution of specific frequency sub-bands including extra-low frequencies (0-10 mHz). These differences have been related to abnormalities in the salience, attentional and default-mode networks functioning, but the inconsistences observed across many studies point to a large heterogeneity in spontaneous brain activity in ADHD. Despite this, evidence suggests that some characteristics of ADHD brain activity are sensitive to specific frequency bands.

Computer-aided diagnosis of attention-deficit/hyperactivity disorder (ADHD) aims to provide useful adjunctive indicators to support more accurate and cost-effective clinical decisions. Deep machine-learning (ML) techniques are increasingly used to identify neuroimaging-based features for objective assessment of ADHD. Despite promising results in diagnostic prediction, substantial barriers still hamper the translation of the research into daily clinic. Few studies have focused on functional near-infrared spectroscopy (fNIRS) data to differentiate ADHD condition at the individual level.

It is therefore an objective of the present disclosure providing a computer-implemented method for selecting functional biomarkers to identify a target condition in a subject, wherein said target condition may be, for example, attention-deficit/hyperactivity disorder (ADHD).

It is also an objective of the present disclosure providing a classifier being trained using the selected functional biomarkers, so it may be used for classifying contributing to the objective assessment of target conditions such as ADHD through computer-aided affordable tools deployable in many clinical settings.

### SUMMARY

According to an embodiment of the disclosure, there is provided a computer implemented method for selecting functional biomarkers to identify a target condition, such as attention-deficit/hyperactivity disorder in a subject, includes obtaining groups of equivalent physiological signals generated during a stimulation, including a first group of physiological signals from subjects presenting the target condition, and a second group of physiological signals from subjects of control condition, not presenting the target condition; identifying at least one frequency sub-band for each group of physiological signals. The frequency sub-band can be a single frequency sub-band, for example a frequency sub-band between 0.0025 Hz and 0.145 Hz or encompassing the whole frequency of a scalogram of the first and second group of physiological signals. The frequency sub bands can also be a plurality of frequency sub-bands corresponding to frequencies that show higher group synchronization; obtaining per each identified frequency sub-band a corresponding pattern, the pattern can be a time series pattern, in the domain of time, or a frequency series pattern, it is a spectral pattern, in the domain of frequency; and selecting one or more patterns having similarity values to the physiological signals of each group that differentiate the groups of physiological signals as functional biomarkers to identify the target condition, for example for training a classifier of the target condition for identifying the target condition from a physiological signal obtained from a subject during the same stimulation.

According to an advantageous aspect, the computer implemented method allows selecting patterns, time series patterns or frequency series patterns, as functional biomarkers to develop a methodological approach for effective identification of a target condition in a subject, such as training a classifier, for example a machine learning classifier of those known in the state of the art. This classifier may then be used in a computer implemented method for identifying if a subject from which an equivalent physiological signal is obtained during the same stimulation is more likely to be a subject presenting the target condition or a subject not presenting the target condition, it is, a subject presenting the control condition.

According to an embodiment of the disclosure, identifying at least a frequency sub-band comprises identifying a plurality of frequency sub-bands for each of the first group and the second group, the plurality of frequency sub bands corresponding to frequencies that show a higher group synchronization than other frequencies; and the obtaining, for each of the at least one frequency sub band, at least a corresponding pattern comprises obtaining, for each of the plurality of frequency sub bands, a corresponding time series pattern; and selecting, as functional biomarkers to identify the target condition, comprises selecting, as functional biomarkers to identify the target condition, one or more of the time series patterns having similarity values corresponding to the first physiological signals of the first group or the second physiological signals of the second group that differentiate the first physiological signals of the first group and the second physiological signals of the second group.

According to an embodiment of the disclosure, the stimulation is a periodical task having a task frequency, the frequency sub-bands including the task frequency.

According to an embodiment of the disclosure, the physiological signal is one of a NIRS signal, a functional magnetic resonance imaging-blood-oxygen-level-dependent (fMRI-BOLD) signal, or an electroencephalography (EEG) signal, or a hemodynamic signal such as a blood pressure or flow signal. The method may also be performed simultaneously for different physiological signals, therefore obtaining functional biomarkers corresponding to physiological signals of different kind. Moreover, the selected functional biomarkers may be combined for training the classifier.

According to an embodiment of the disclosure, the physiological signal is a cerebral signal, obtained in a same region of the brain, in response to relative or absolute changes in the concentration of a hemoglobin chromophore, so the cerebral signal may be externally obtained with devices known by the skilled person.

According to an embodiment of the disclosure, the identifying the frequency sub-band includes, per each group: obtaining a time-frequency transform of each physiological signal; averaging the time-frequency transforms; extracting the local-maxima of the averaged time-frequency transforms; and identifying the frequency sub-bands for the group as frequency ranges around the local-maxima using a suitable predefined criteria such as between the local-minima around each local-maxima. Other suitable predefined criteria could also be used, such as setting a percentage of height descent on both sides around the local-maxima, for example, 50% of the local-maxima height. The physiological signals are expected to be adapted as necessary, and also signals obtained from the physiological signals, such as transforms, may also be used. The number of frequency sub-bands may also be different per each group. For example, frequency sub-bands could be limited to those presenting a bandwidth within a certain threshold, or frequency sub-bands with a local-maximum within a certain threshold, or selecting a predefined number of frequency sub-bands based on the best bandwidth and local-maxima values. It is also envisaged, when using multiple physiological signals, to combine frequency sub-bands obtained from multiple physiological signals in case several physiological signals of similar frequency response are used, such as similar cerebral signals obtained from different regions of interest, so the common most relevant frequency sub-bands are identified. For example, averaging frequency sub-bands or selecting the combined maxima of the frequency sub-bands.

According to an embodiment of the disclosure, the time-frequency transform is a continuous wavelet transform. Other transforms known by the skilled person, such as short-timed Fourier transform, or Wigner-Wille transform could also be used.

According to an embodiment of the disclosure, the identifying the frequency sub-bands includes calculating an inter-subject synchronization measure, and identifying the frequency sub-bands for the group as frequency ranges around the local-maxima. The inter-subject synchronization measure is one of a phase-synchronization measures, or coherence measures, or correlation measures, of the measures known by the skilled person.

According to an embodiment of the disclosure, the inter-subject synchronization measure includes a phase synchronization measure.

According to an embodiment of the disclosure, the inter-subject synchronization measure includes a combination of a phase and magnitude synchronization measure.

According to an embodiment of the disclosure, the obtaining per each identified frequency sub-band a corresponding time-series pattern per each group includes: computing the inverse time-frequency transform from the calculated time-frequency transform of the physiological signals per each frequency sub-band to obtain the corresponding, band-limited, time-series for each member of the group; and generating the time-series pattern of the group by averaging the obtained time-series for each member of the group.

According to an embodiment of the disclosure, the step of selecting the one or more time-series patterns that better differentiate the groups of physiological signals includes calculating time-series similarity measures, such as Euclidean, Mahalanobis, Cityblock distances or elastic measures, as Dynamic Time Warping, or correlation methods. Similarity is calculated between each candidate time-series pattern and the time-series of each member of the group, and selecting the time-series patterns with similarity measures that better separate the groups, as functional biomarkers.

According to an embodiment of the disclosure, the best time-series patterns are selected based on statistical contrast at a predefined significance level, such as the F-statistic for the analysis of variance, so only the most relevant time-series patterns are selected, thus improving the computational efficiency of the classifier. Other selection criteria of the best time-series patterns are also envisaged, even no selection is contemplated, using all available time-series patterns.

According to an embodiment of the disclosure, the target condition may be one of: a target mental condition, target brain disorder condition, target body physiological condition, or a combination of them. For example, the target condition may be one of: attention-deficit/hyperactivity disorder, mild-cognitive impairment, neurodegenerative disorders as Alzheimer's disease, dysautonomia disorders, depression, or anxiety. However, other target conditions may also be envisaged.

According to another embodiment of the disclosure, there is provided a classifier for identifying the target condition in a subject trained using the similarity values to the functional biomarkers. The classifier using the selected time-series patterns as functional biomarkers allows classifying if a physiological signal of a subject is likely to be a physiological signal from a subject presenting the target condition or from subjects of control condition, not presenting the target condition.

According to another embodiment of the disclosure, there is provided a computer implemented method for identifying the target condition in a subject using the trained classifier, the method includes obtaining the equivalent physiological signal from a subject during the same stimulation previously used for selecting the functional biomarkers; and processing the physiological signal in the classifier for identifying the target condition in the subject. Also, according to another embodiment of the disclosure, there is provided computer programs including instructions that when executed cause a machine to perform the previous computer implemented methods.

According to another embodiment of the disclosure, there is provided a method of diagnostic of the target condition in a subject using the computer implemented method described above. For example, once the classifier is trained for identifying the target condition, the target condition can be detected in any subject. The target condition may be a target mental condition, target brain disorder condition, target body physiological condition, or a combination of them, such as attention-deficit/hyperactivity disorder, mild-cognitive impairment, neurodegenerative disorders as Alzheimer's disease, dysautonomia disorders, depression, or anxiety,

### BRIEF DESCRIPTION OF DRAWINGS

As a complement to the description provided herein and for the purpose of helping to make the characteristics of the disclosure more readily understandable, this specification is accompanied by a set of drawings, which by the way of illustration and not limitation, represent the following:

FIG. 1 presents a general schema of the computer implemented method of the present disclosure for selecting functional biomarkers F;

FIG. 2 presents a general schema of the computer implemented method of the present disclosure for training a classifier using the selected functional biomarkers F.

FIG. 3 presents a general schema of the computer implemented method of the present disclosure detecting a target condition in a subject using the trained classifier.

FIG. 4 presents a schematic of a stimulation or task for obtaining physiological signals from a subject;

FIG. 5 presents a setup for obtaining physiological signals from a subject, in this case, the physiological signals corresponding to cerebral signals from three regions of interest, right, medial, left using a known device;

FIG. 6 presents a physiological signal obtained from a subject and the corresponding continuous wavelet transform (CWT) of the physiological signal.

FIG. 7a presents the schematics of the procedure to compute a representative inter-subject synchronization (ISS) map, from physiological signals of a same group of subjects, presenting o not presenting the target condition;

FIG. 7b presents the inter-subject synchronization ISS of FIG. 7a and its frequency sub-bands;

FIG. 8 presents an schematic example of the procedure to extract a candidate time-series pattern from the physiological signals of a group of subjects, and comparing the candidate time-series pattern with time-series obtained from the physiological signals of all groups of subjects for determining if the candidate time-series pattern differentiates the groups of physiological signals;

FIG. 9a presents the inter-subject synchronization of the physiological signals for shallow-signals for the three regions of interest, right, medial, left obtained with the setup of FIG. 5;

FIG. 9b presents inter-subject synchronization of the physiological signals for clean-signals CS for the three regions of interest, right, medial, left obtained with the setup of FIG. 5;

FIG. 10 presents the average inter-subject synchronization patterns across frequencies of the three regions of interest, for shallow-signals and clean-signals, for HbO and HbR chromophores of the group of subjects presenting the target condition and the group of subjects not presenting the target condition, and the corresponding frequency sub-bands.

FIG. 11a presents time-series pattern candidates for shallow signals (SS) for each frequency sub-band, each region of interest (right, medial, left), and each chromophore (HbO -upper-, HbR -lower-) from the group of subjects presenting the target condition and the group of subjects not presenting the target condition;

FIG. 11b presents time-series pattern candidates for clean signals (CS) for each frequency sub-band, each region of interest (right, medial, left), and each chromophore (HbO -upper-, HbR -lower-) from the group of subjects presenting the target condition and the group of subjects not presenting the target condition; and

FIG. 12 presents performance scores achieved by each classification model trained using the selected time-series patterns.

FIG. 13 presents a general schema of another embodiment of the computer implemented method of the present disclosure for selecting functional biomarkers F.

FIG. 14 presents an example of processing of a physiological signal in the another embodiment of the computer implemented method.

FIGS. 15a and 15b presents an example of obtaining frequency series patterns in the another embodiment of the computer implemented method.

FIG. 16 presents a general schema of the another embodiment of computer implemented method of the present disclosure for training a classifier using the selected functional biomarkers F.

FIG. 17 presents a general schema of the another embodiment of the computer implemented method of the present disclosure detecting a target condition in a subject using the trained classifier.

### DETAILED DESCRIPTION

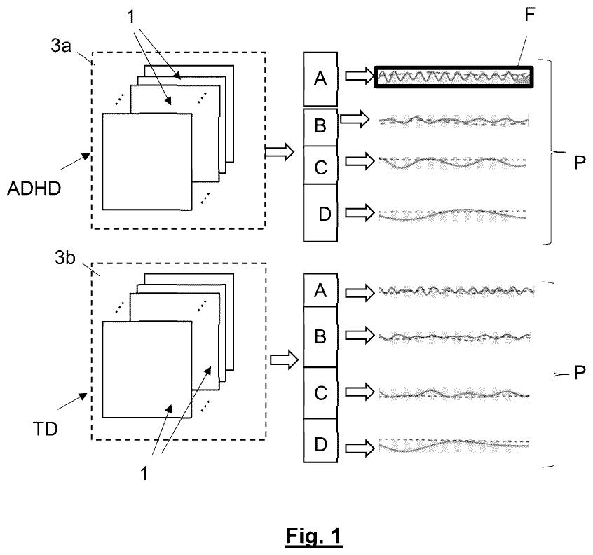

FIG. 1 presents a general schema of the computer implemented method according to an embodiment of the disclosure for selecting functional biomarkers F to identify a target condition ADHD in a subject. As it can be seen, the method includes obtaining groups of equivalent physiological signals 1, generated during a stimulation S, including a first group 3a of physiological signals 1 from subjects presenting the target condition ADHD, and a second group 3b of physiological signals 1 from subjects of control condition TD, not presenting the target condition ADHD. For example, the first group 3a of physiological signals may be signals obtained from performing measurements on one or more subjects identified as having a particular condition and the second group 3b of physiological signals may be signals obtained from performing measurements on one or more subjects identified as not having the particular condition. According to an example embodiment, the particular condition may be ADHD, but the disclosure is not limited thereto. According to an example embodiment, the one or more subjects may be identified as having the particular condition based on a diagnosis performed on the one or more subject. As it will be later described, the stimulation S may be a periodical task having a task frequency, the frequency sub-band A, B, C, D including the task frequency. The physiological signal 1 may be a cerebral signal, such as a NIRS signal, a fMRI-BOLD signal, or an EEG signal, or an hemodynamic signal such as a blood pressure or flow signal. As it will be later shown, the physiological signal 1 may be a cerebral signal, obtained in a same region of the brain, in response to relative or absolute changes in the concentration of a hemoglobin chromophore. It is expected that the method described could be programmed and/or stored in a memory and executed by a computer, for example a local, remote, distributed computer or an embedded device. Further, a program including instructions that when executed cause a machine to perform the methods described are envisaged.

As shown in FIG. 1, the method may include, for each of the first group 3a and the second group 3b of physiological signals 1, identifying frequency sub-bands A, B, C, D corresponding to frequencies that show higher group synchronization. Therefore, each of the first group 3a and the second group 3b will be associated with certain frequency sub-bands A, B, C, D. As also shown, the method further includes obtaining per each identified frequency sub-band A, B, C, D, of each of the first group 3a and the second group 3b a corresponding time-series pattern P. Although four frequency sub-bands A, B, C, D are illustrated in FIG. 1, the disclosure is not limited thereto, and as such, according to another embodiment, frequency sub-bands less than or greater than four may be identified. Also, a single frequency sub-band is envisaged, for example encompassing the frequencies with higher energy distribution.

Once a time-series pattern P is obtained per each frequency sub-band A, B, C, D of each of the first group 3a and the second group 3b, the method further includes selecting the one or more representative time-series patterns P having similarity values d to the physiological signals 1 of each group that differentiate the groups of physiological signals 1 as functional biomarkers F to identify a target condition ADHD, that may be used for training a classifier CL of the target condition ADHD.

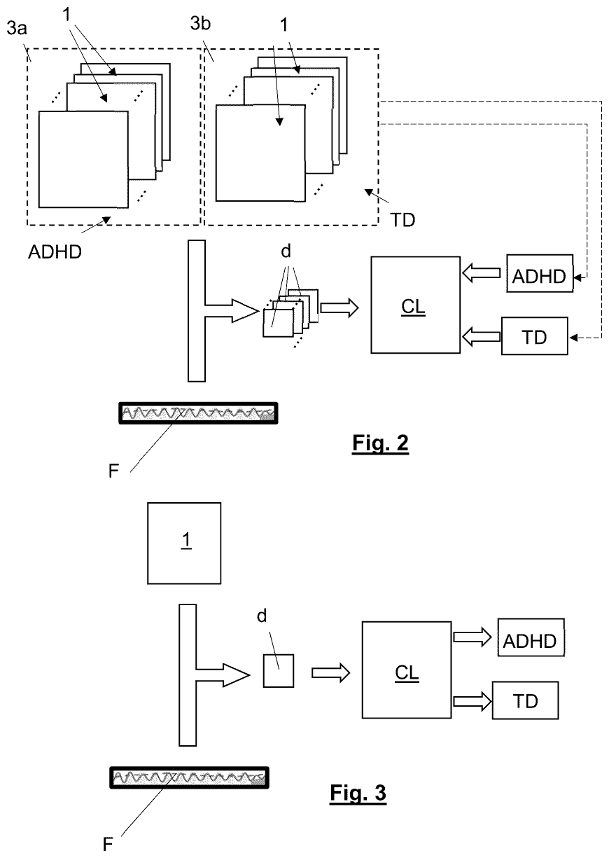

FIG. 2 presents a schema of the training of a classifier CL for the target condition ADHD and control condition TD using the similarity values d to the selected functional biomarkers F of the previous physiological signals 1 of each of the first group 3a and the second group 3b. The classifier CL is trained using supervised learning, each similarity value d being fed to the classifier CL indicating whether the similarity value d corresponds to a physiological signal 1 of the first group 3a, presenting the target condition ADHD, or to a physiological signal 1 of the second group 3b of a control condition TD not presenting the target condition ADHD. This information may be fed as a probability of the physiological signal 1 being of the first group 3a, thus having the target condition ADHD.

FIG. 3 presents a schema of a computer implemented method for identifying a target condition ADHD in a subject, which includes obtaining a physiological signal 1 from a subject during a stimulation S, and processing the physiological signal 1 in the classifier CL for identifying the target condition ADHD in the subject. The physiological signal 1 and the stimulation S must be equivalent to the physiological signal 1 and stimulation S used during training, so the obtained physiological signal 1 from the subject 2 may be classified. For example, the output of the classifier CL may be a probability of the physiological signal 1 being of the first group 3a, thus having the target condition ADHD. If this probability is higher than a threshold, also a binary output may be given, thus informing that the subject is likely to show the target condition ADHD.

A detailed embodiment related to a computer implemented method for selecting functional biomarkers to identify a target condition of ADHD in a subject will be explained thereafter. Naturally, other target mental or brain disorder conditions, target body physiological conditions or combination of them could be used as a target condition for selecting the corresponding functional biomarkers for that target condition. Therefore, the target condition may be a target mental condition, target brain disorder condition, target body physiological condition, or a combination of them, such as attention-deficit/hyperactivity disorder, depression, or anxiety.

In this embodiment, groups of equivalent physiological signals generated during a stimulation S will be obtained. For example, a first group 3a of physiological signals 1 from one or more subjects presenting the target condition ADHD, and a second group 3b of physiological signals from one or more subjects of control condition TD, not presenting the target condition may be obtained. Although a single equivalent physiological signal could be used, in the described embodiment several equivalent physiological signals will be used, so functional biomarkers could be extracted from each of the equivalent physiological signals. According to an example embodiment, equivalent physiological signals may mean same type of kind of signals. For example, in the embodiment, two kind cerebral signals (shallow-signal SS and clean-signal CS), from three different regions of interest (left, medium, central) and corresponding to the concentration of two chromophores, therefore, twelve equivalent physiological signals will be used for obtaining the functional biomarkers F for each equivalent signal that separate the group of subjects presenting the target condition (ADHD) and the group of subjects of control condition (TD), not presenting the target condition.

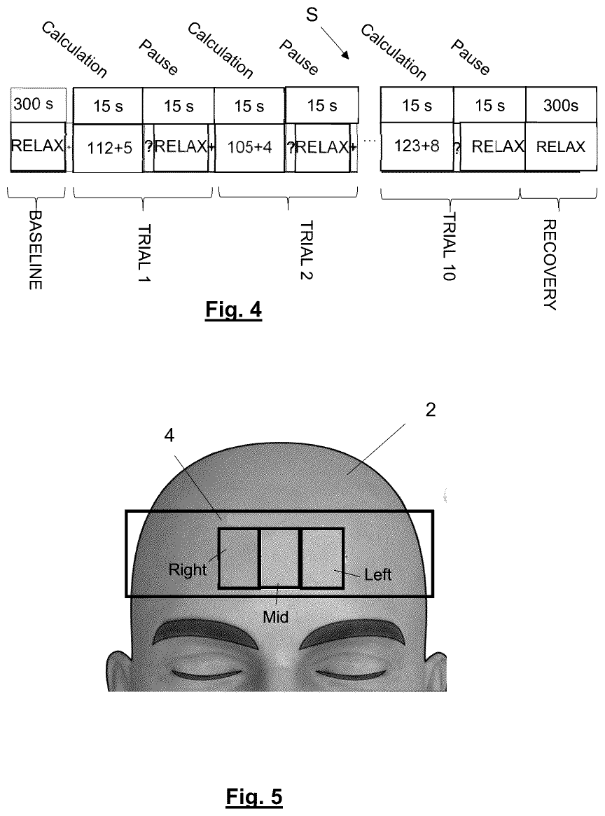

FIG. 4 presents an example of a stimulation that may be used for the computer implemented method for selecting functional biomarkers to identify a target condition according to an embodiment of the disclosure. In this case this stimulation is a mental or cognitive activity that may be programmed in a computer including 10 consecutive 30 seconds trials, each starting with 15 seconds of mental calculation followed by a 15 seconds pause of relaxation. During mental math, participants were asked to iteratively add a small number (5 to 9) to a three-digit number (100 to 199) (both numbers randomly chosen), silently and as quickly and accurately as possible. The pause then begins by presenting the question “Result?” for 5 seconds, prompting for the voicing of the final result reached, followed by a black screen indicating mental relaxation until a 2 seconds fixation cross announced the start of the next trial. The task lasted 300 seconds and was uninterruptedly preceded by 300 seconds of baseline recording in resting state and followed by another 300 seconds of recovery in relaxed state. The 30 seconds period of the trials corresponds to a frequency of 0.033 Hz, which will be referred to as the task frequency. This design also minimizes speech during the task, thus avoiding significant changes in breathing that could affect cerebral hemodynamics. Although an example of the simulation is illustrated for understanding, the disclosure is not limited thereto, and as such, other stimulations could be used instead according to other example embodiments, as long as they may be reproduced for obtaining the physiological signals when selecting the functional biomarkers and when classifying using the trained classifier. According to an example embodiment, even non periodical tasks of stimulations could be used as long as they may be reproduced. According to an example embodiment, not only mental tasks are considered, also physical tasks are contemplated, for example body tilting or physical exercises, that could also be periodic, as periodically raising an arm.

According to an example embodiment, for recording and processing the physiological signal, as a fNIRS signal, analysis may be performed on superficial and regression-corrected deep fNIRS signals recorded from the forehead of a subject 2 through a multi-distance, multi-channel device 4. According to an example embodiment, a device 4 as illustrated in FIG. 5 may be similar to the device from Tehia, Newmanbrain, S. L., Elche, Spain also described in Molina-Rodriguez S et al. (2022, “Frequency-domain analysis of fNIRS fluctuations induced by rhythmic mental arithmetic Psychophysiology”).

Using such device 4, relative concentration changes in oxy- (HbO) and deoxy-hemoglobin (HbR) may be computed for different regions of interest. For example, relative concentration changes in oxy- (HbO) and deoxy-hemoglobin (HbR) may be computed for a left region, a middle region and a right region. Therefore, physiological signals may be external cerebral signals from three regions of interest (ROI), right, medial, left, to different chromophores HbO HbR and different signal type (shallow-signal-SS-and clean-signal -CS-), it is, twelve physiological signals may be considered. However, the disclosure is not limited to the example regions illustrated in FIG. 5.

According to an example embodiment, identification of the frequency components with potential capacity to differentiate between the two groups of participants (i.e., having the control condition TD and having the target condition ADHD) may be one of the features for identifying ADHD in a subject, and will be discussed in detail below.

To this end, a suitable method for locating stimulation or task-related oscillations may be used on different time scales (i.e., frequency bands), appropriate for non-stationary signal analysis, and capable of providing some measure of similarity to define class membership. A data-driven approach based on time-frequency transform, as complex continuous wavelet transform (CWT), and time-scale synchronization detection may be used.

The CWT is an time-frequency transform signal processing method that provides a time-frequency (or time-scale) representation of the characteristics of a signal base on the dilation and translation of a mother wavelet function. CWT may be viewed as a bandpass filter with varying bandwidths automatically defined by the wavelet scale, which avoids the drawbacks of using custom filters.

According to an example embodiment, to compute the complex continuous wavelet transform CWT generalized Morse wavelets may be used. For example, a flexible superfamily of exactly analytic wavelets particularly useful for analyzing signals with time-varying amplitude and frequency, i.e. modulated signals, may be used. Since Morse wavelets may be tuned to encompass many other analytic wavelets commonly used, Morse wavelets provide a unified framework as reference point.

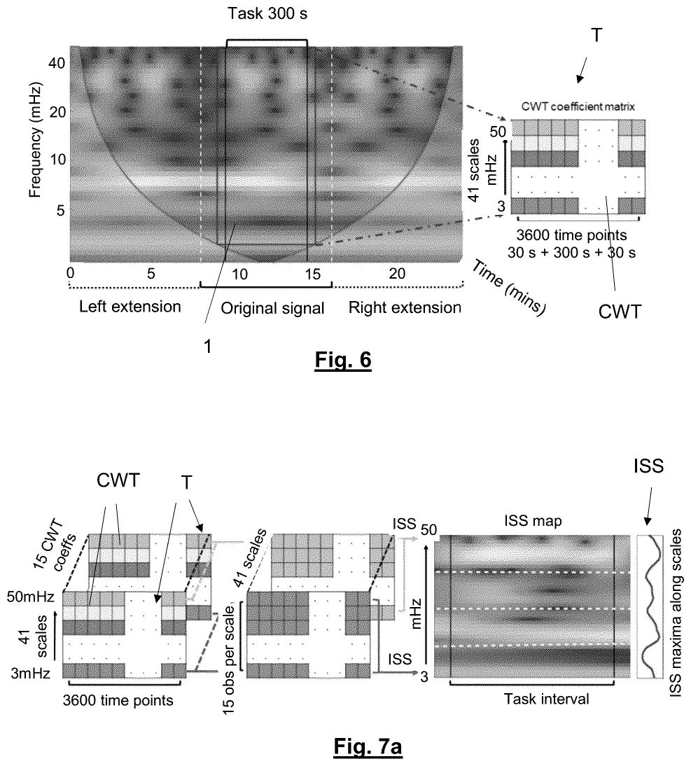

Depending on the kind of physiological signal, the range of frequencies of interest may be adapted, for example for NIRS or fMIR being up to 0 to 100 mHz; EEG being up to 0 to 40 Hz; and electromyogram being up to 0 to 600 Hz. Also the range of frequencies of interest may be determined by the Nyquist frequency of the sampling rate, as known by the skilled person. In the example embodiment as illustrated in FIG. 6, a preliminary PSD analysis shows that most of the spectral power is within the 0-50 mHz band, and the CWT is focused on that frequency range. For example, the CWT may be applied to the symmetrically extended signals with the scale discretization parameter voices-per-octave=10, which after calculation of the minimum and maximum allowable bandpass results in 45 scales with approximate frequencies ranging from 2.4 to 50 mHz. Thus, in this case the number of usable scales is limited to 41 (3 to 50 mHz), as illustrated in FIG. 6.

FIG. 6 shows a first part of identifying frequency sub-band A, B, C, D per each of the first group 3a and the second group 3b. In FIG. 4 it can be seen that a time-frequency transform T is obtained of each physiological signal 1, as a continuous wavelet transform (CWT). FIG. 6 shown a schematic of a continuous wavelet transform CWT of deep HbO signal from mid-ROI. Also FIG. 6 presents a scalogram of the symmetrically extended data with the cone-of-influence. White dashed lines enclose the original physiological signal and the solid black lines delimit the task-period. A box depicts the scalogram region from which the coefficients for analysis were extracted. Further schematics of the extracted coefficient matrix containing 41 scales and 3600 time points corresponding to the box is presented. After CWT, the full coefficient matrix is kept for later computation of the inverse CWT (i.e. including the data corresponding to the extended segments). According to an example embodiment, only a shorter portion of the matrix may be used for the later computation. Under the hypothesis that the stimulation, as a math task, may induce differentiated fNIRS fluctuations for the control condition TD and target condition ADHD groups, the frequency sub-bands that showed higher group synchronizations during the task may be identified.

Since group-wise synchronization may appear as transient peaks rather than constantly, a time-point-by-time-point analysis may be performed, which allows capturing common oscillatory patterns that evolve dynamically over time. For example, an inter-subject correlation (ISC) analysis may be performed, which is a data-driven approach for assessing consistent neural responses to stimuli across.

According to an example embodiment, instantaneous inter-subject synchronization (ISS) may be measured using the magnitude and phase information provided by the complex-valued CWT coefficients. In fMRI studies, measures as inter-subject phase synchronization and pairwise phase consistency have been validated for the assessment of voxel-wise instantaneous phase synchronization across subjects. However, these measures rely only on the uniformity of phase angles, ignoring the magnitude. Thus, when applied to fNIRS, it may mean that low-amplitude signals affect the measurement the same as significant amplitude signals (or high amplitude signals). Therefore, this approach may not be entirely appropriated for fNIRS data where amplitude changes are related to the magnitude of the hemodynamic response. Since as amplitude increases, the signal-to-noise ratio improves, it is reasonable to argue that observations with higher amplitudes may contribute to a more realistic estimate of phase synchronization. As such, according to an example embodiment, an ‘inter-trial linear coherence’ measure may be used, which combines magnitude and phase in the normalization operation. Since the measurement here was across subject observations and not across trials, this measurement may be referred to as ‘inter-subject synchronization’ (ISS) measure throughout the disclosure. Moreover, the ISS measure may omit ‘linear’ for simplicity and similarly. However, the disclosure is not limited thereto. According to an embodiment, the ISS measure may include a combination of a phase and magnitude synchronization measure, formulated as:

$$ISS ( f , t ) = \frac{{\sum }_{k = 1}^{n} {F}_{k} ( f , t )}{\sqrt{n {\sum }_{k = 1}^{n} {\vert \text{?} ( f , t ) \vert}^{2}}} ,$$

$$\text{?} \text{indicates text missing or illegible when filed}$$

where Fₖ (f,t) is the spectral estimate of observation k at frequency f and time t, and the modulus∥ represents the complex norm. ISS also takes values between 0 (absence of sync) and 1 (perfect sync).

The ISS may be computed moment-to-moment for each scale from the CWTs coefficient matrices of each group (i.e. 15 participant observations per scale). The analysis may be limited to only to the time-interval between −30 seconds and +30 seconds around the task. However, the disclosure is not limited thereto, and as such, the parameters for computation may be varied according to other example embodiments. According to an embodiment, the ISS procedure may be applied independently to the shallow-signal SS and clean-signal CS data of each group of subjects for each chromophore and ROI. However, the disclosure is not limited thereto, and as such, the ISS procedure may be applied in various manner. An ISS representation in the time-frequency plane that may be visualized as a ISS map is illustrated in FIG. 7a.

The maximum ISS observed along each scale may be chosen, which represents the highest group synchronization achieved at each specific frequency, as can be also seen in FIG. 7a.

FIG. 7a, shows a part of the identifying frequency sub-band A, B, C, D per each of the first group 3a and the second group 3b, once a time-frequency transform T as a continuous wavelet transform CWT is obtained for each physiological signal 1. This part of identifying frequency sub-band method may include averaging the time-frequency transforms T of the first group 3a and the second group 3b, and calculating an inter-subject synchronization ISS measure that may be a phase synchronization measure, and also a combination of a phase and magnitude synchronization measure.

Therefore, in FIG. 7a. schematics of the procedure to compute a representative inter-subject synchronization (ISS) map may be observed. Starting with the coefficient matrices of each individual within a group (left), complex-valued data are put together for each frequency bin (middle) and the ISS is computed moment-to-moment to obtain a representation of the within-group synchronization strength at each frequency over time (ISS map). The rightmost plot (ISS maxima along scales) depicts the maxima and minima reached by the ISS along frequencies, which are used to delimit the sub-bands to analyze (horizontal white dashed lines), shown in FIG. 7b.

As shown in FIG. 7b, the local-maxima Lmax is extracted from the averaged time-frequency transforms T of the first group 3a and the second group 3b, calculated as the inter-subject synchronization ISS, and the frequency sub-bands A, B, C, D for the group are identified as frequency ranges around the local-maxima Lmax, in this case between local-minima Lmin around a local-maximum Lmax.

Once frequency sub-bands A, B, C, D are identified, the computer implemented method may further include obtaining per each identified frequency sub-band A, B, C, D a corresponding time-series pattern P and selecting the one or more representative time-series patterns P having similarity values d to the physiological signals 1 of each group that differentiate the groups of physiological signals 1 as functional biomarkers F to identify the target condition ADHD, and that may be used during the training of a classifier CL of the target condition ADHD. For example, the method may include computing an inverse time-frequency transform IT from the calculated time-frequency transform T of the physiological signals 1 per each frequency sub-band A, B, C, D to obtain a corresponding time-series t for each member of the group, it is, for each subject of the first group 3a and the second group 3b, and generating a time-series pattern P of the group by averaging the obtained inverse time-frequency transform IT for each member of the group.

Once one or more time-series patterns P are obtained, selecting the one or more time-series patterns P that better differentiate the groups of physiological signals 1 may include calculating time-series similarity values d between each candidate time-series pattern P and the time-series t of each member of the group, and selecting the time-series patterns P with similarity values d that better differentiate the groups as functional biomarkers F. The time-series patterns P may be selected as functional biomarkers F based on statistical contrast at a predefined significance level.

Like the other aforementioned synchronization measures, ISS is a compound measure that does not exist on its own at a single-subject level but represents a summary statistic of group synchronization. Therefore, to disentangle the contribution to ISS of each individual is not a straightforward issue.

However, ISS peaks suggest that some frequency components show similar time courses across individuals, at least within certain time intervals. In other words, there are sequential patterns common to the group that may provide distinctive information to define class membership. This concept may be referred to as time-series classification, which encompasses a variety of techniques for identifying those properties (features) that have sufficient differentiating power to distinguish between different classes of time series. In the context of the example embodiment of the disclosure, a well-suited technique could be the one based on the shapelet framework, which addresses the classification problem by discovering primitive time-series sequences (shapelets) that are used to quantify the similarity between classes of time series. Shapelets provide directly interpretable information about patterns (shapes) that are important for understanding how data classes differ, a desirable property for clinical decision support systems.

According to an example embodiment, a basic shapelets technique may be applied. However, instead of looking for phase-independent subsequences similar in shape (i.e. subsequences may be located anywhere in the series), the analysis within a fixed time-interval may be performed, all subsequences having the same length. According to an example embodiment, subsequence translation over time may not applied. This may mean that time-series similarity also depends on the phase (i.e. on a consistent time-alignment). Therefore, instead of local patterns, global patterns present over a whole time interval may be captured. Under this approach, the term “time-series pattern” may be used instead of “shapelet”. However, the disclosure is not limited to the use of this term.

According to an example embodiment, the method may further include extracting the time-series to be used for identifying representative time-series patterns P. The average ISS patterns suggest the frequencies that are likely to contain synchronized oscillations. By computing the inverse time-frequency transform IT as inverse CWT within the specific frequency sub-band A, B, C, D defined by the bounds of an ISS peak, a band-limited components in the time-domain may be reconstructed. To reduce edge-effects, the inverse CWT may be computed from the extended coefficient matrix that was reserved in a previous operation. Then, the resulting time-series were truncated to the interval between −30 seconds and +30 seconds around the task. After applying this procedure to the CWT of all the individuals belonging to a group, a set of time-series (n=15) may be obtained to find a reference time-series pattern for that group in a particular sub-band.

Since all the time-series have the same length and are within the same timeframe, a suitable reference time-series pattern may be obtained simply by averaging. If the time-series share a common pattern, their average should represent the group well enough. To quantify similarity with the reference time-series pattern, among other possibilities, a simple measure as Euclidean distance may be computed:

$$D ( S , T ) = \sqrt{\text{?} {( \text{?} - \text{?} )}^{2}}$$

$$\text{?} \text{indicates text missing or illegible when filed}$$

where S denotes the reference time-series pattern and T a time-series, both of length n. Note that S and T should be standardized to have mean 0 and standard deviation 1, which ensures to operate on the same scale. Standardization also allows us to relate the Euclidean distance to the correlation coefficient (r=1-D²/2n).

In order to assess the capability of the time-series pattern to differentiate between groups, the distances obtained from one group with those of the other group were contrasted. For example, a time-series pattern that is representative of the control condition TD group should have smaller distances to members of this group than to members of the target condition ADHD group, and vice versa.

Among other quality measures, the F-statistic for analysis of variance may be used to assess the discriminative power of a time-series pattern. This statistic indicates the ratio of the between-group variability to the within-group variability as:

$$F = \frac{\text{?} \frac{{( {\overline{D}}_{i} - \overline{D} )}^{2}}{C - 1}}{\text{?} {\sum }_{{d}_{j}} ∈ {D}_{i} \frac{{( {d}_{j} - {\overline{D}}_{i} )}^{2}}{n - C}}$$

$$\text{?} \text{indicates text missing or illegible when filed}$$

where C is the number of classes (or groups; for example, C=2), D is the overall mean of all distances, [symbol]
   is the mean of the distances within class i, and n is the number of time-series. The better the time-series pattern the greater the F value, because the difference between-groups increases while it decreases within a group. The corresponding p-value may be calculated from the F-distribution.

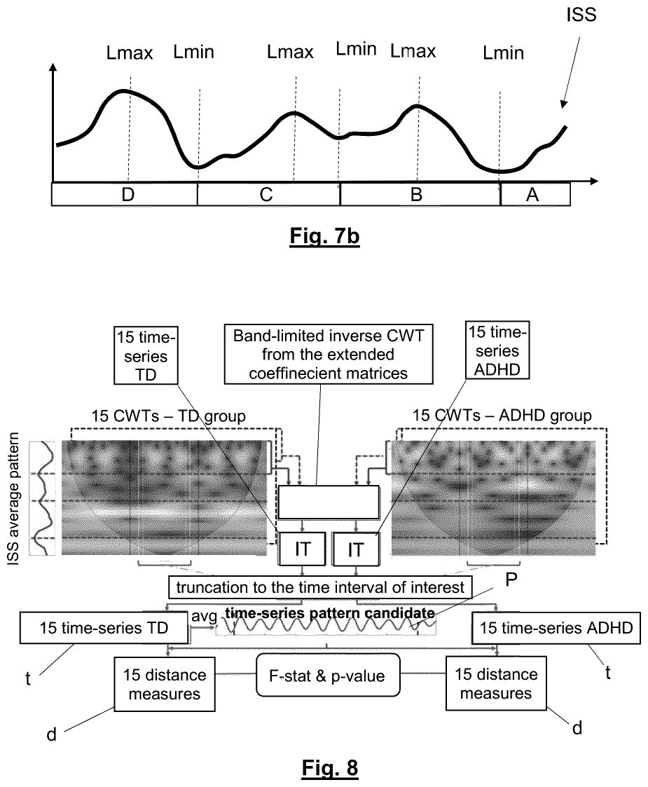

According to an example embodiment, based on the average ISS patterns, four candidate frequency sub-bands A, B, C, D may be identified in each of them that were labeled A, B, C and D in decreasing order of frequency. However, the disclosure is not limited thereto, and as such, candidates of frequency sub-bands may be different than four. Each sub-band may contain a peak (local-maximum) flanked by two troughs (local-minima) that delimit the frequency boundaries. For each ISS pattern, each belonging to a target group (TD or ADHD), the following procedure for each sub-band may be performed, as indicated in FIG. 8: (i) Compute the inverse CWT within the sub-band from the extended coefficient matrices of both groups to obtain the corresponding time-series (n=15+15=30). (ii) Truncate time-series to the time-interval of interest. (iii) Generate the reference time-series pattern by averaging only the time-series of the target group (n=15), a variability measure such as the standard error of the mean (SEM) may also be computed. (iv) Calculate distances to the time-series pattern for the time-series of both groups. (v) Calculate F and p-value from the 15+15=30 distances. Because each ISS pattern and its sub-bands are common to all three ROIs, this procedure may be applied separately to each ROI data but using the same sub-bands. Thus, for each ISS pattern a matrix of 30×12 distances may be obtained, where each row contains the distance measures of an individual and columns correspond to the 4 sub-bands ×3 ROIs. Since there are eight ISS patterns, the final matrix was of size 30×96, 48 columns for SS and 48 for CS, while 15 rows correspond to the TD group and 15 to ADHD. Each column in the matrix may have an associated F-stat and p-value, indicating how well a particular time-series pattern differentiates the groups.

According to an example embodiment, the time-series pattern approach is illustrated to transform data observations at different time-scales into a simple feature space of Euclidean distances, but the disclosure is not limited thereto, and as such, other feature type approaches in the state of the art could be used to transform data observations without deviating from the example embodiment of the disclosure.

FIG. 8 shows a schematic example of the procedure to extract reference time-series patterns for the control condition TD group and contrast them with the target condition ADHD group. Per-subject time-series within a specific frequency sub-band A, B, C, D are obtained from their band-limited inverse-CWT and averaged only for the control condition TD group to get the time-series pattern. Euclidean distances to the time-series pattern are computed for the time-series of both groups and then within- and between-group variances are contrasted to obtain the F-stat value.

To assess the feasibility of the procedure to differentiate between control condition TD and target condition ADHD, four well-suited classifiers CL, machine learning algorithms for supervised binary classification, namely linear support vector machine (SVM), logistic regression (LR), linear discriminant analysis (LDA) and Gaussian naïve Bayes (NB) were tested.

These algorithms were selected because they are well known, inherently interpretable, computationally efficient, and may work with relatively small sample sizes. Under a variety of flavors (different kernel, regularization, etc.), SVM is very frequently present in neuroimaging-based studies of brain disorders, with LDA and LR being the other most popular choices. According to an embodiment, NB may be included due to its ease of application and good performance in a variety of applications despite the assumption of feature independence.

SVM differentiate classes by finding the hyperplane that maximizes the separation (margin) between the points of them. LR do the job through a logistic function (sigmoid) that model the dependent variable and maps predicted values into probabilities of belonging to one class or another. LDA assumes that the predictors come from a Gaussian mixture distribution and uses discriminant functions to estimate the probability that they are from each class. NB estimates the probability density of predictors given a class by independently mapping them onto separate normal distributions fitted to each class. Although based on different models, discriminative (LR & SVM) vs generative (LDA & NB), all four are within the linear classifier category, i.e. to make predictions, the classifiers try to learn the line that best separates the points of the two classes.

In addition to the putative functional response, fNIRS signals also contain components originating from common systemic forces and unpredictable local activity. Therefore, it is very likely that the feature matrix also contains redundant and/or irrelevant data that may degrade classifier performance by cause of overfitting and noise issues. Model regularization may be applied to some algorithms to account for statistical overfitting, however that raises the problem of choosing a suitable technique (e.g. lasso) and finding appropriate regularization parameters. To avoid increasing the complexity of the models, the problem may be addressed by reducing the feature space. Feature selection is a commonly used tool to obtain a smaller subset of the most relevant features, reducing complexity while improving classification accuracy and generalization capacity. A reasonable hybrid approach is to first apply a filter method, before modeling, to select some features based only on their intrinsic properties. Then more sophisticated methods such as wrappers may be employed to find the best subset of features, using the classifier itself as evaluator. Among others, a benefit of such selection is an easier explanation of the prediction because the models are simpler.

A wide variety of filter methods have been developed, each based on specific criteria (information, similarity, etc.) to evaluate features. According to an example embodiment, the filter may be selected based on the F-statistic. For example, the features that showed p-values<0.01 was selected for testing purpose, assuming that their generating time-series patterns were very unlikely to separate the groups by chance. In this way, the features may be significantly reduced from 48 to 5 for SS and from 48 to 10 for CS. However, the disclosure is not limited thereto, and as such, according to another embodiment, filters may be selected based on relief, minimum redundancy-maximum relevance, chi-square, etc.

According to an example embodiment, the classification methods may be first applied to filter-selected features separately for SS and CS, and then for all of them together (SS+CS). To assess the predictive performance, two cross-validation (CV) techniques for comparison purposes were applied, namely leave-one-out (LOO) and stratified 5-fold. In the first, data was partitioned into 30 folds where each observation was used once as a test set and the remaining ones formed the training set. In the latter, five partitions were randomly chosen, each with 26 observations as the training set and 6 as the test set; folds were repartitioned over 20 Monte-Carlo repetitions (5×20=100 models) to reduce CV variance, while stratification ensured that sets had the same proportion of classes (50% in an example case). Also, 5-fold instead of 10-fold was used because with the latter the test set size=3 would be too close to that of LOO=1. Moreover, since only two classes and well-balanced datasets (i.e. equal proportion of both classes) are used, accuracy, specificity and sensitivity may be used as metrics to assess performance, as obtained from the corresponding confusion matrices and then averaged across folds. At this point, accuracy (a commonly used metric in practice) was the considered to test the statistically significant classification performance. Thus, the theoretical above-chance accuracy threshold based on the binomial cumulative distribution at p<10-3 for 2-classes (probability=0.5) and a sample size=30 was computed.

Afterwards, a wrapper method may be applied to fine-tune the feature selection such as a Sequential Forward Floating Selection (SFFS) algorithm. SFFS starts with an empty set and sequentially adds one feature at a time to create candidate subsets that are evaluated by cross-validation. After that, the best feature is added to the set. When the size of the selected set is >2, a backward step tries to optimize it by removing one or more features. This procedure is repeated until there is no performance improvement. The two aforementioned cross-validation techniques were also applied independently to each classifier. Noteworthy, the input order of the finally selected features allows us to know their relative importance. According to an embodiment, the wrapper can rank features by multiple metrics at the same time, and as such, two criteria may be used to select/remove features, specificity and then accuracy. Thus, if two (or more) features equally improve the specificity, the one with the best accuracy is selected.

Once the best subset of features was selected for each classifier, the statistical significance of the observed performance may be estimated through a non-parametric label permutation procedure that does not assume any particular statistical property of the data. For example, for the testing, 5000 resamples were generated, each of which randomly permuted the labels of the two classes; realizing the null hypothesis that features do not define class membership. For each resampled data, the classification performance was evaluated using the same cross-validation scheme as for the real data. The observed performance metrics were ranked against the corresponding null-distribution to estimate a p-value. In addition, a 95% bias corrected percentile interval was estimated as confidence interval for each metric by bootstrapping (with replacement) over 2000 resamples, with each realization keeping the same proportion of classes (50%) and at least three distinct observations in each class.

In addition, according to an embodiment, the testing may include checking whether the wrapper performance might have been biased due to the use of a pre- filtered feature set that included all data in the selection process, i.e. the “peeking” effect. To this end, during the testing, repeated the wrapper procedure was repeated using the full feature set (i.e. 96 features), then comparing the performance outcomes.

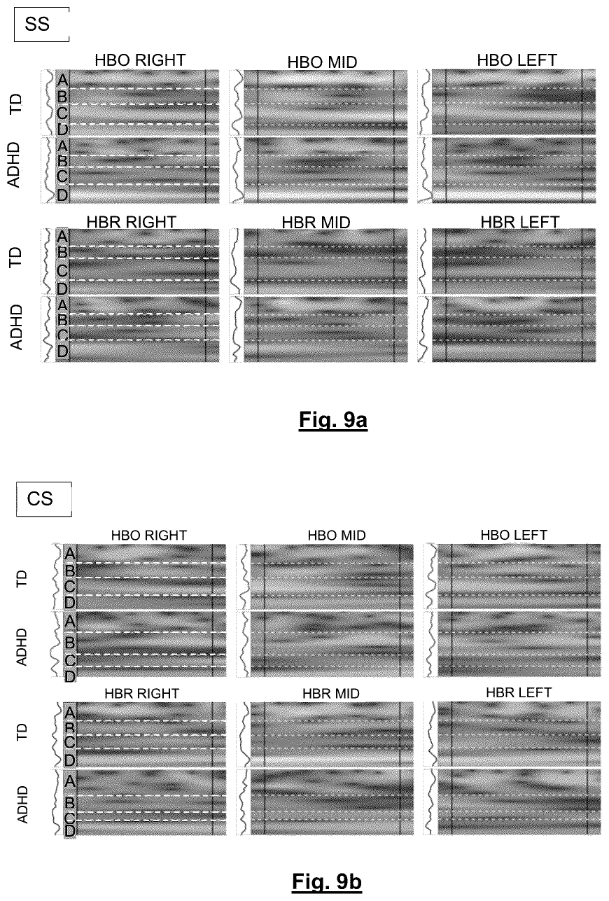

FIG. 9a shows the ISS representation in the time-frequency plane obtained from the complex CWTs of the SS data for both groups; within the range of 3 to 50 mHz. Similarly, FIG. 9b depicts the ISS maps corresponding to the CS data. At first glance, well-defined synchronization zones may be seen within certain frequency sub-bands, some similar in both groups and others clearly differentiated. Here some of them as examples are highlighted. Regarding SS, strong synchronization may be seen in all ROIs around 33 mHz for HbO of TD group, and in right- and mid-ROI at 7 mHz and bellow 4 mHz. Noteworthy, the ADHD group presents even stronger ISS around 4 mHz in all ROIs, but much less evident at 7 or 33 mHz. Regarding CS, albeit to a lesser extent, TD group also synchronizes at 33 mHz while TD group does so in a more diffuse and weak way. Furthermore, TD group seems to be more synchronized during the first part of the task at 7 mHz (mid- and left-ROI), whereas the ADHD group is synchronized in the last part around 17 mHz. Yet another remarkable sync is observed for the HbR of TD group at 4 mHz. Overall, ISS analysis reveals a plurality of sub-bands that may carry information about similarities and differences between groups.

FIG. 9a represents inter-subject synchronization (ISS) maps for shallow-signals SS within each ROI. Upper rows correspond to HbO for TD and ADHD groups, while lower rows refer to HbR. The small plots to the left of each map depict the ISS maxima across frequencies for each case. Vertical black lines delimit the task-interval. Horizontal white dashed lines delimit the common sub-bands obtained by averaging ISS maxima across ROIs. Labels A-D identify each of these sub-bands. FIG. 9b represents inter-subject synchronization (ISS) maps for clean-signals CS in a similar manner that in FIG. 9a.

Based on the observed local-maxima Lmax, on each ISS map of FIGS. 9a and 9b, several peaks may be identified within specific frequency sub-bands A, B, C, D that contain oscillatory components showing some synchronization at the group level. Such components may be expected to provide information to differentiate between groups, and as such, frequency sub-bands A, B, C, D exhibiting more discriminative power may be identified or located.

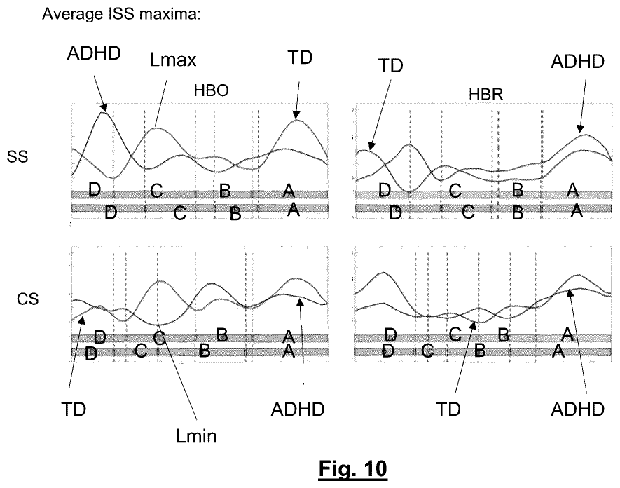

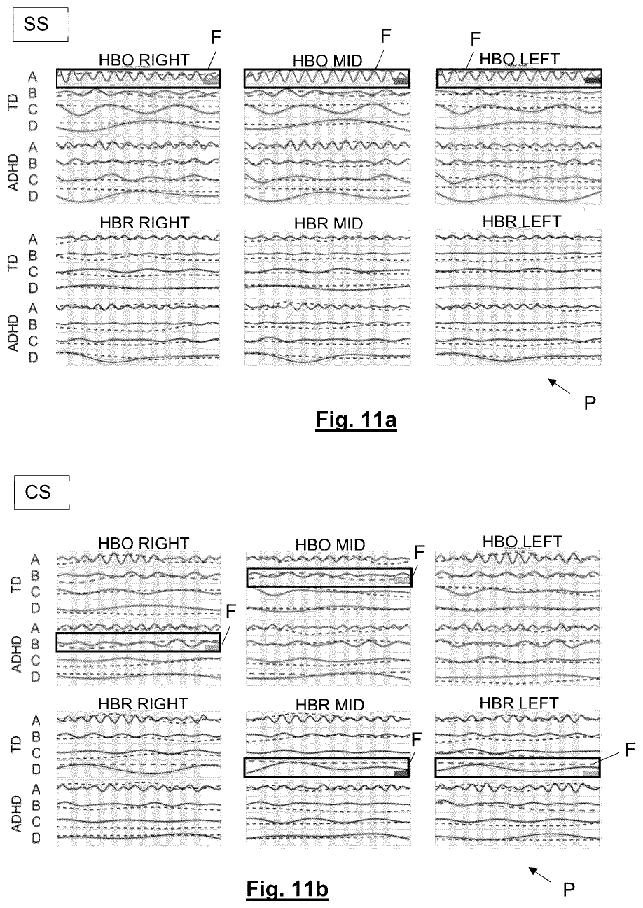

As illustrated in FIG. 10, the procedure may be simplified, by averaging, for each case, the ISS data across the three ROIs to obtain the mean ISS maxima per frequency. Thus, the analysis may be simplified to only one common ISS pattern by chromophore and signal type for each of the two groups, i.e. 2 groups (TD, ADHD)×2 chromophores (HbO, HbR)×2 signal types (SS, CS)=8 ISS patterns. Finally, to reduce noise, the ISS patterns may be smoothed by moving average using a sliding window of length half the voices-per-octave (i.e. 10/2=5). These patterns may be examined or analyzed in the following operations to identify the most relevant frequency components.

Table 1 shows the common synchronization sub-bands estimated from the average ISS maxima across ROIs, labeled A, B, C and D by decreasing frequency, with A corresponding to the task frequency.

TABLE 1
Frequency bounds of each average sub-band A to D for shallow-signals
(SS) and clean-signals (CS), and for each chromophore and group.

| Signal | | | Frequency sub-band (mHz) | | | |
|---|---|---|---|---|---|---|
| type | Chromophore | Group | A | B | C | D |
| SS | HbO | TD | 50-21.8 | 21.8-11.7 | 11.7-4.7 | 4.7-3.0 |
| | | ADHD | 50-23.3 | 23.3-14.3 | 14.3-6.7 | 6.7-3.0 |
| | HbR | TD | 50-23.0 | 23.0-14.3 | 14.3-5.4 | 5.4-3.0 |
| | | ADHD | 50-23.0 | 23.0-14.3 | 14.3-7.7 | 7.7-3.0 |
| CS | HbO | TD | 50-21.8 | 21.8-11.7 | 11.7-5.4 | 5.4-3.0 |
| | | ADHD | 50-20.3 | 20.3-7.7 | 7.7-4.7 | 4.7-3.0 |
| | HbR | TD | 50-21.8 | 21.8-11.7 | 11.7-6.7 | 6.7-3.0 |
| | | ADHD | 50-16.5 | 16.5-8.2 | 8.2-5.8 | 5.8-3.0 |

FIG. 10 illustrates how these frequency sub-bands A, B, C, D were delineated by locating the ISS local-minima Lmin surrounding each peak or local-maximum Lmax. Higher peaks may be seen in sub-bands A and C for HbO of TD group in both SS and CS, while ADHD group shows notable peaks in D for SS and B for CS. Regarding HbR, it shows clear peaks in A and D in all cases. Note that within the same assigned sub-band, in some cases the peaks are clearly shifted in frequency depending on the group (e.g. C sub-band for CS-HbO). It is evident again that the ISS maxima also reveal differences at certain frequencies. FIG. 10 therefore illustrates the average inter-subject synchronization (ISS) patterns across frequencies. The top row corresponds to shallow-signals (SS) and bottom row to clean-signals (CS). Left and right traces relate respectively to HbO and HbR, with the TD group data and ADHD data. Horizontal labeled rectangles identify each frequency sub-band A, B, C, D, whose boundaries are defined by the local-minima Lmin marked by vertical dashed lines.

FIG. 11a shows the reference time-series patterns obtained in each sub-band for SS data and FIG. 11b those corresponding to CS data. A rich variety of patterns may be seen, some similar across groups and others clearly different. Thus, for example, TD group exhibits rhythmic fluctuations in the A sub-band of SS-HbO, which are very consistent across participants as reflected by the high ISS (dashed traces); in contrast, ADHD group shows greater inter-subject variability. Another example is visible in CS-HbR-D, were TD group shows a consistent pattern of increasing then decreasing, whereas ADHD group does not. It may also be seen that the ADHD group synchronizes CS-HbO-B towards the end of the task, while TD does so visibly earlier. Once again, certain time-series patterns P seem to represent well the average response of their group, while they do not fit the other one.

Table 2 shows the performance achieved by classifiers CL trained with the features, the functional biomarkers as selected time-series patterns, which were selected by filtering, i.e. those with an F p-value<0.01.

TABLE 2
Performance scores achieved by each classification model trained with the subset of
filter-selected features for shallow-signals (SS), clean-signals (CS) and SS + CS.

| Signal | | SVM | | LR | | LDA | | NB | |
|---|---|---|---|---|---|---|---|---|---|
| type | P. metric | 5-FO | LOO | 5-FO | LOO | 5-FO | LOO | 5-FO | LOO |
| SS | Accuracy | 73.3 | 70 | 80 | 80 | 74.5 | 73.3 | 80.5 | 80 |
| 5 features | Sensitivity | 78.7 | 73.3 | 85.7 | 86.7 | 80.7 | 80 | 88.7 | 86.7 |
| | Specificity | 78.7 | 73.3 | 85.7 | 86.7 | 80.7 | 80 | 88.7 | 86.7 |
| CS | Accuracy | 81.2 | 80 | 83.5 | 80 | 78.7 | 80 | 85.5 | 86.7 |
| 10 features | Sensitivity | 82.3 | 80 | 82 | 80 | 81.7 | 80 | 84.7 | 86.7 |
| | Specificity | 82.3 | 80 | 82 | 80 | 81.7 | 80 | 84.7 | 86.7 |
| CS & SS | Accuracy | 88.7 | 86.7 | 88.2 | 90 | 85.7 | 83.3 | 85.7 | 86.7 |
| 15 features | Sensitivity | 91.3 | 93.3 | 91.7 | 93.3 | 82.7 | 73.3 | 84 | 85.7 |
| | Specificity | 86 | 80 | 84.7 | 86.7 | 88.7 | 93.3 | 87.3 | 86.7 |

Note:
LOO = leave-one-out; 5-FO = 5 folds; SVM = support vector machine; LR = logistic regression, LDA = linear discriminant analysis; NB = naive Bayes.

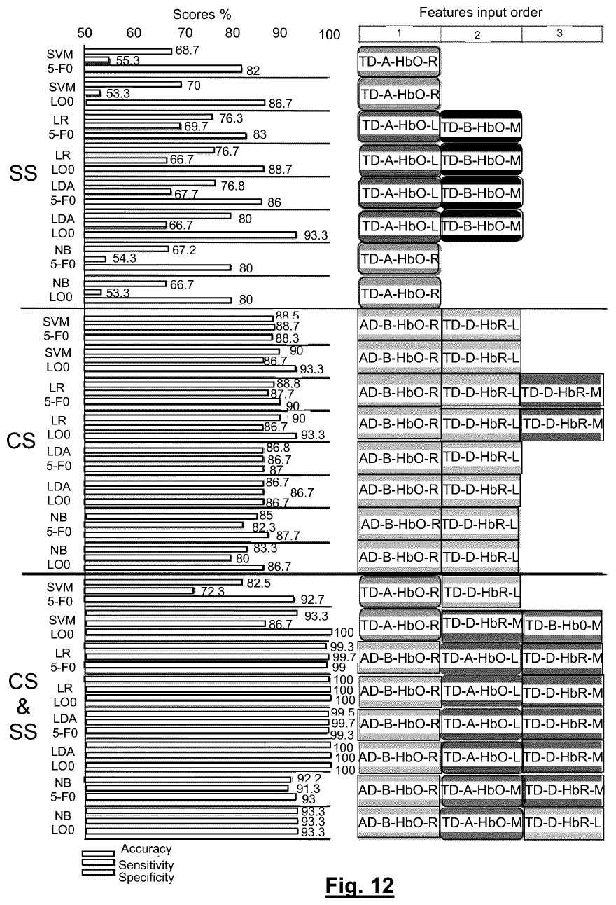

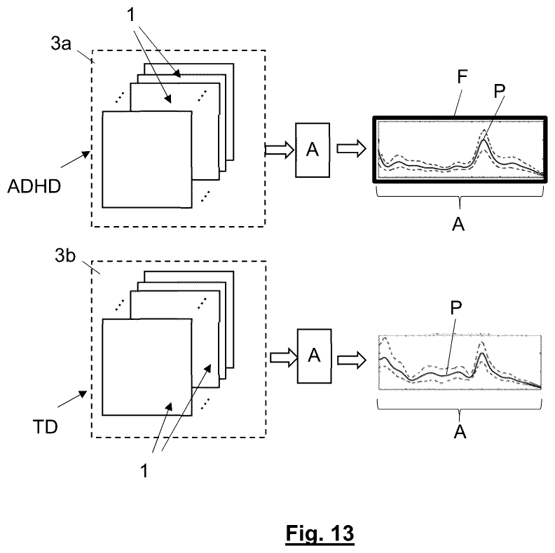

FIG. 11a presents shallow-signals (SS) reference time-series patterns P for each frequency sub-band A, B, C, D within each ROI. Upper rows show the HbO patterns for TD and ADHD groups, while lower rows refer to HbR; thin traces depict the time-series pattern's SEM. Dashed lines show the inter-subject synchronization (ISS) time-course in each frequency sub-band A, B, C, D (scale on the right axis); shaded region corresponding to time-series patterns with an F p-value<0.01. The boxes identify the time-series patterns P chosen by the wrappers as functional biomarkers F (see FIG. 12). Gray shaded rectangles indicate each of the 15 seconds of mental math during stimulation S task. FIG. 11b presents clean-signals (CS) reference time-series patterns P with a description of the drawn elements being the same as in FIG. 11a.

FIG. 12 demonstrates how performance was greatly improved by using the wrapper for feature selection. Note that, in all cases, the classification models were very parsimonious and no more than three features were used. When compared separately, wrapper-selected CS features perform better that SS overall. Regarding the input order, for SS the first-in feature always belong to TD group, A sub-band, HbO, left- or right-ROI depending on the classifier (“TD-A-HbO-L” or “TD-A-HbO-R” in FIG. 9). It should be noted, regarding CS, that all the classifiers agree on the first two features “AD-B-HbO-R” and “TD-D-HbR-L” (see FIG. 12). Once again, the best results were obtained with SS+CS. In fact, LR and LDA scored over 99% on all three metrics for both cross-validation schemes, which is really high performance. NB scored lower, from 91.3% to 93.3% overall while SVM was the weakest classifier when all metrics are considered.

FIG. 12 presents performance scores achieved by each classification model using the subset of wrapper-selected features for shallow-signals (SS), clean-signals (CS) and SS+CS. Rectangles at the right identify the chosen features and their input order. Each specific feature has a label, which is the combination “Group-Band-Chromophore-ROI”, where TD=TD group, AD=ADHD group, L=left, M=mid and R=right.

When evaluating SS+CS, the wrapper selected the same features for LR and LDA in both cross-validation cases. Noteworthy, the first-in feature was “AD-B-HbO-R”, which was also the first for CS. The second one was “TD-A-HbO-L”, the first for SS. Finally, “TD-D-HbR-M” from CS completed the set. Looking at the wrapper history, “AD-B-HbO-R” alone achieves about 80% of the accuracy, sensitivity and specificity, which is not surprising since it has the highest F (19.6, p-val<0.0001). The addition of “TD-A-HbO-L” improves the scores up to 90% and with “TD-D-HbR-M” they approach 100%. Therefore, the most powerful feature comes from the CS-HbO data of ADHD group, specifically from band B in which a prominent ISS peak may be seen (FIG. 11b). TD group provides the next best feature in form of consistent HbO fluctuations at task frequency in the SS data (see the ISS peak in sub-band A). Finally, a very-slow CS-HbR component of TD group optimizes the classification (see the peak in D). The time-series patterns that generated these features are respectively identified by boxes in FIGS. 11a and 11b. Noteworthy, NB also shared the first feature while the second and third come from the same group, sub-band and chromophore but from an adjacent ROI.

Table 3 summarizes the results obtained by wrappers with SS+CS features. In all cases (except for SVM in sensitivity with 5-fold) permutation testing indicated significance at p<0.001. LR and LDA showed the highest scores in all metrics and also the narrowest CIs. Although highly significant, NB showed lower performance and larger ICs, whereas SVM performed the worst.

TABLE 3
Performance scores achieved by each classification model using the best subset of wrapper-selected
features obtained from shallow- (SS) plus clean-signals (CS). Statistical significance is
indicated by p-values and performance 95% CIs are represented within square brackets.

| | | SVM | | LR | | LDA | | NB | |
|---|---|---|---|---|---|---|---|---|---|
| Signal | P. metric | 5-FO | LO0 | 5-FO | LOO | 5-FO | LOO | 5-FO | LOO |
| CS | Accuracy | 82.5 | 93.3 | [symbol] | 100 | 99.5 | [symbol] | 92.2 | 93.3 |
| & | | [symbol] 001 | p < .001 | p < .001 | p < .001 | p < .001 | p < [symbol] | p < 001 | p < 001 |
| SS | | [68.2, 95.7] | [80.0, 100} | [95.8, 100] | [ [symbol] , 100] | [96.7, 100] | [ [symbol] , 100] | [80.3, 98.0] | [ [symbol] , 100] |
| | Sensitivity | 72.3 | 86.7 | 99.7 | 100 | 99.7 | 100 | 91.3 | [symbol] |
| | | p = .046 | p < .001 | p < .001 | p < .001 | p < .001 | p < .001 | p < .001 | p < .001 |
| | | [ [symbol] , 73.3] | [ [symbol] ] | [ [symbol] , 100] | [ [symbol] , 100] | [ [symbol] , 100] | [93.3, 100] | [73.3, [symbol] ] | [ [symbol] , 100] |
| | Specificity | 92.7 | 100 | [symbol] | 100 | [symbol] | 100 | [symbol] | [symbol] |
| | | p [symbol] .001 | p < .001 | p < .001 | p < .001 | p < .001 | p < .001 | p < .001 | p < .001 |
| | | [78.2, 100] | [ [symbol] , 100] | [ [symbol] , 100] | [ [symbol] , 100] | [ [symbol] , 100] | [ [symbol] , 100] | [80, 100} | [80.0, 100] |
| | Features | 2 | [symbol] | 3 | 3 | 3 | 3 | 3 | 3 |

Note:
LOO = leave-one-out: 5-FD = 5-fold; SVM = support vector machine; LR = logistic regression; LDA = linear discriminant analysis; MB=naive Bayes. [symbol]
indicates data missing or illegible when filed

According to an embodiment, when the full feature set (48 SS+48 CS) was used to feed the wrappers for LR and LDA, the same feature subset for both cross-validation schemes may be obtained, and as such, the same scores and statistics may be obtained. Therefore, feature pre-selection may provide good performance with reduced computational cost (i.e., lower computer resources such as memory and processing resources).

Surprisingly, SVM performed the worst in the SS+CS case, particularly with the 5-fold cross-validation where it achieved the lowest scores and also the widest CIs. In contrast, the other classifiers were not affected by the cross-validation, reaching similar scores and CIs with both schemes (Table 3). Most likely, SVM performance degraded due to lack of proper regularization, leading to insufficient penalty for misclassification.

As seen from FIG. 12 and Table 3, LR and LDA won in the classification task when trained with SS+CS features. They both agreed on the same wrapper solution, which was exactly the same for the features preselected by filtering and for the full set. Also, they achieved comparable scores (>99%) in all metrics (significant at p<0.001, but now using permutation tests to define the null hypothesis). In addition, the 95% CIs were similarly narrow, although skewed due to scores being close to the 100% ceiling. Cross-validation is known to tend towards narrower confidence bounds as accuracy approaches 100% but increasingly wide and asymmetric as sample size decreases, which may lead to under-estimate prediction errors and specially with LOO cross-validation. Despite having only 30 samples, the lower limit of the CI for accuracy was never <95.8% and was less than 5% away from the mean in all cases, a deviation below the overall 15% expected for a binary classification with this number of samples. The results suggest that the few selected features meet the statistical assumptions required by LR and LDA to make observations highly separable into 2-classes. The rest of this discussion focuses on LR and LDA and their three wrapper-selected shared features.

LOO and k-fold are the most commonly adopted cross-validation methods in ADHD studies; plus hold-out which seems more appropriate for large datasets. Some researchers have suggested that LOO may be more useful in a diagnostic scenario, whereas others recommend k-fold or repeated random splits for more stable estimates. In either case, it is known that cross-validation is compromised by small sample sizes, particularly if there are many predictors, which tend to overestimate predictive accuracy to a variable degree depending on the particularities of the study. In light of this, LOO and 5-fold were tested expecting differences in performance, with LOO showing more optimistic scores and larger confidence bounds. However, both LOO and 5-fold produced very similar results with LR and LDA (Table 3), suggesting that the three chosen features are good predictors to yield stable cross-validation measures regardless of method.

According to an embodiment illustrated in FIGS. 13 to 17, the selected functional biomarkers to identify the target condition are one or more frequency series patterns. As shown in FIG. 13, the method may include, for each of the first group 3a and the second group 3b of physiological signals 1, identifying at least one frequency sub-band A. In this case one frequency sub-band corresponding to the range of frequencies from 0.0025 Hz to 0.145 Hz is identified. This range of frequencies includes the oscillations caused by the cognitive task and the oscillations related to myogenic, neurogenic and endothelial activity. However, the usage of other ranges of frequencies and more frequency sub-bands are envisaged. In this embodiment, both of the first group 3a and the second group 3b will be associated with the same frequency sub-band A. As also shown, the method further includes obtaining per the identified frequency sub-band A of each of the first group 3a and the second group 3b, which is the same frequency sub-band A, a corresponding pattern P, in this embodiment a frequency series pattern P, also known as spectral pattern, which can be the average of the power of each frequency in the frequency sub-band A and within a time interval for the physiological signals 1 of each subject 2 of the first group 3a, and a frequency series pattern P can be the average of the power of each frequency in the frequency sub-band A and within a time interval for the physiological signals 1 of each subject 2 of the second group 3b. Although only one frequency sub-band A is illustrated in FIG. 13, as previously explained, the disclosure is not limited thereto, and as such, according to other embodiments, frequency sub-bands greater than one may be identified. Note that the frequency series patterns P represented also include in dashed line a confidence interval, corresponding to 95%

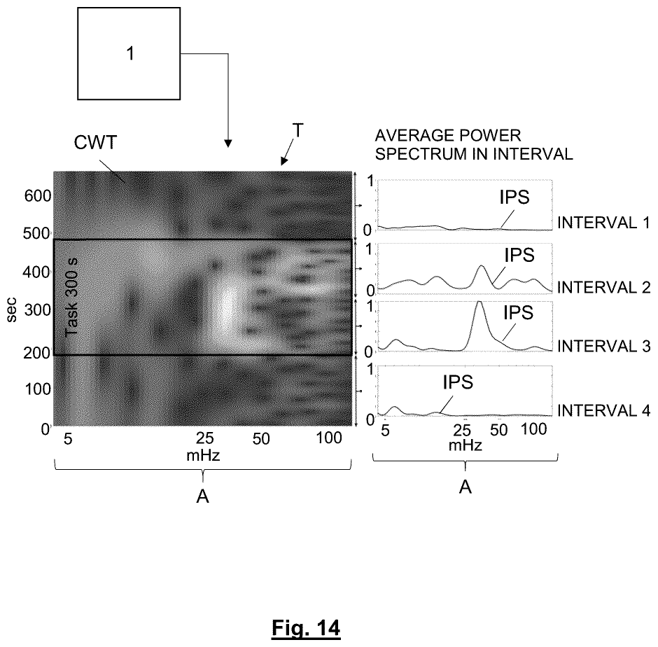

FIG. 14 illustrates an example of generating four interval power spectrum (IPS) signals from a physiological signal 1 from the subject 2 of one group 3a, 3b that will be later averaged with other power spectrum IPS signals from other subjects 2 of the same group 3a, 3b to obtain a frequency series pattern P for a given interval for the given group 3a, 3b. As shown in FIG. 14, for each physiological signal 1 a time-frequency transform T is applied, obtaining the spectrum of the physiological signal 1 within a time range, which can be the same time range of the physiological signal 1. This spectrum is also called scalogram. The scalogram may be normalized so the maximum power is 1. The time-frequency transform T can be for example a continuous wavelet transform CWT using as mother wavelet function a complex Morlet function, but other time-frequency transforms as FFT or other wavelet functions for the continuous wavelet transform can be used alternatively. The time range can be divided into intervals, for example four intervals as presented in FIG. 14. For each interval, the interval power spectrum IPS is calculated by averaging the power spectrum for the interval. A single interval or more intervals are also envisaged. The shown time intervals INTERVAL 2 and INTERVAL 3 correspond to the cognitive task previously presented, therefore it is expected that the time intervals corresponding to the cognitive task will be the more representative for obtaining the functional biomarkers F. Although functional biomarkers F could be obtained from other intervals, in the following example only INTERVAL 3 will be considered for illustrating an aspect of the disclosure.

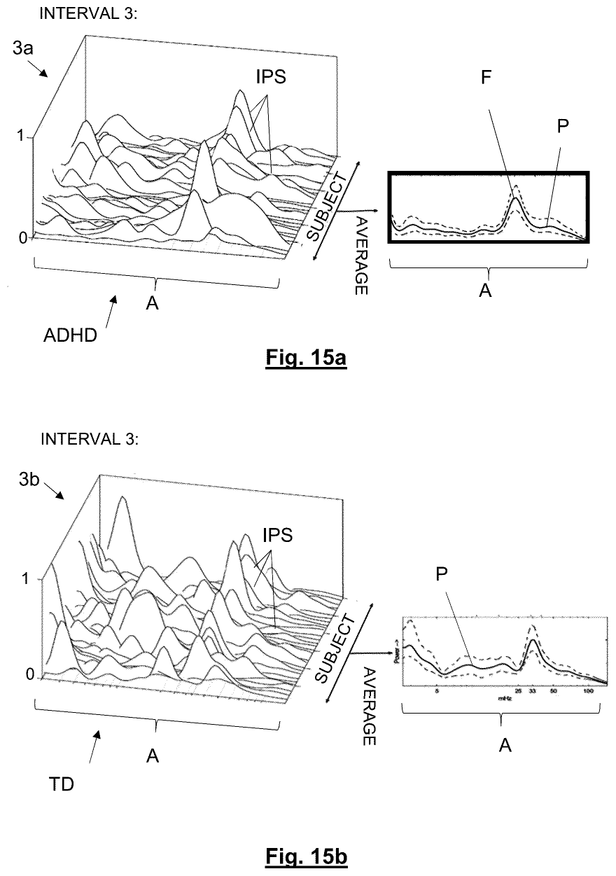

FIGS. 15a and 15b show the generation of the frequency series pattern P corresponding to the first group 3a for INTERVAL 3 and sub-band A, by averaging the interval power spectrum IPS corresponding to each subject 2 of the first group 3a, and the generation of the frequency series pattern P corresponding to the second group 3b for INTERVAL 3 and sub-band A, by averaging the interval power spectrum IPS corresponding to each subject 2 of the second group 3b. Each frequency series pattern P can be considered a spectral signature for their respective group 3a, 3b.

Once a frequency series pattern P, a spectral pattern, is obtained per the frequency sub-band A for each of the first group 3a and the second group 3b, the method further includes selecting the one or more representative frequency series patterns P having similarity values d to the physiological signals 1 of each group that differentiate the groups of physiological signals 1 as functional biomarker F to identify a target condition ADHD, that may be used for training a classifier CL of the target condition ADHD. As previously explained the similarity value d can be the Euclidian distance.

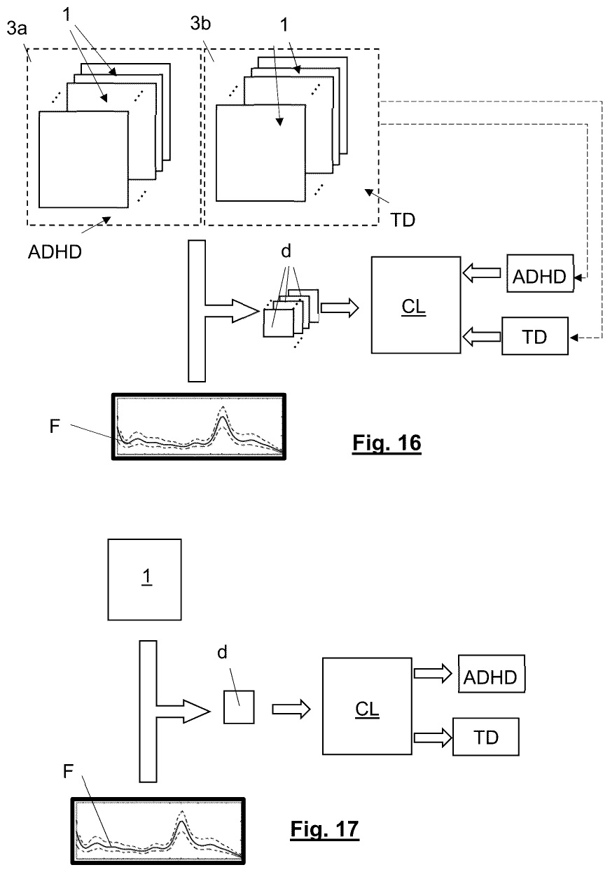

Similarly, as previously explained in FIG. 2, FIG. 16 illustrates a classifier CL that is trained for detecting the target condition ADHD and control condition TD using the similarity values d to the selected functional biomarkers F of the previous physiological signals 1 of each of the first group 3a and the second group 3b. Naturally, as in this case the functional biomarkers F are frequency series patterns, the similarity values d will be calculated based on the time-frequency transform T of the physiological signals 1, it is from their interval power spectrum IPS signal for the time interval. The classifier CL, which can be based for example on logistic regression or support vector machines, is trained using supervised learning, each similarity value d being fed to the classifier CL indicating whether the similarity value d corresponds to a physiological signal 1 of the first group 3a, presenting the target condition ADHD, or to a physiological signal 1 of the second group 3b of a control condition TD not presenting the target condition ADHD. This information may be fed as a probability of the physiological signal 1 being of the first group 3a, thus having the target condition ADHD.

Once the classifier CL is trained, a computer implemented method for identifying a target condition ADHD in a subject can be executed, in a similar way as previously explained in FIG. 3. For example, as illustrated in FIG. 17, the method may include obtaining a physiological signal 1 from a subject during a stimulation S, and processing the physiological signal 1 in the classifier CL for identifying the target condition ADHD in the subject. The time-frequency transform T may be applied to the physiological signal 1 for obtaining its interval power spectrum IPS for the time interval before feeding the physiological signal 1 to the classifier CL. The physiological signal 1 and the stimulation S must be equivalent to the physiological signal 1 and stimulation S used during training, so the obtained physiological signal 1 from the subject 2 may be classified. For example, the output of the classifier CL may be a probability of the physiological signal 1 being of the first group 3a, thus having the target condition ADHD. If this probability is higher than a threshold, also a binary output may be given, thus informing that the subject is likely to show the target condition ADHD.

As previously explained, it is envisaged that the physiological signals 1 may be external cerebral signals from three regions of interest (ROI), right, medial, left, to different chromophores HbO, HbR, HbT and different signal type (shallow-signal -SS- and clean-signal -CS-) from which a scalogram can be obtained as previously explained. Also combinations of physiological signals 1 from the same user 2 can be used, for example, a scalogram of the crossed spectrum between HbO and Hb of the same region of interested can be used, and the crossed spectrum between the same chromophore (HbO, HbR or HbT) of different region of interest can be used.

According to an example embodiment, methods and operations illustrated above may be implemented in an electronic device or a computer. For instance, the electronic device may include a memory storing one or more instructions, and a processor configured to execute the one or more instructions to: obtain, based on a stimulation procedure, a first group of first physiological signals from one or more first subjects representing a target condition, and a second group of second physiological signals from one or more second subjects representing a control condition not presenting the target condition, identify at least one frequency sub-band for each of the first group and the second group, the at least one frequency sub-band preferably corresponding to one or more frequencies that show a higher group synchronization than other frequencies, obtain, for each of the plurality of frequency sub-bands, at least a corresponding pattern, preferably a time-series pattern, and select, as functional biomarkers to identify the target condition, the one or more patterns, preferably one or more time-series patterns or one or more frequency series patterns, having similarity values corresponding to the first physiological signals of the first group or the second physiological signals of the second group that differentiate the first physiological signals of the first group and the second physiological signals of the second group.

According to an example embodiment, there is provided a non-transitory computer readable medium having stored thereon a computer program including instructions that when executed cause a machine to perform a method including obtaining, based on a stimulation procedure, a first group of first physiological signals from one or more first subjects representing a target condition, and a second group of second physiological signals from one or more second subjects representing a control condition not presenting the target condition, identifying one or more frequency sub-bands for each of the first group and the second group, a plurality of frequency sub-bands may correspond to frequencies that show a higher group synchronization than other frequencies, obtaining, for each frequency sub-band, at least a corresponding pattern, that may be a time series patter or a frequency series pattern, selecting, as functional biomarkers to identify the target condition, one or more patterns having similarity values corresponding to the first physiological signals of the first group or the second physiological signals of the second group that differentiate the first physiological signals of the first group and the second physiological signals of the second group. According to an embodiment, the machine may be a computer or a hardware processor.

While the present disclosure has been described with reference to example embodiments thereof, it will be apparent to those of ordinary skill in the art that various changes and modifications may be made thereto without departing from the spirit and scope of the present disclosure as set forth in the following claims and their equivalents.

What is claimed is:

1. A computer implemented method comprising:
obtaining, based on a stimulation procedure, a first group of first physiological signals from one or more first subjects representing a target condition, and a second group of second physiological signals from one or more second subjects representing a control condition not presenting the target condition; identifying at least one frequency sub-band for each of the first group and the second group;
obtaining, for each of the at least one frequency sub-band, at least a corresponding pattern; and
selecting, as functional biomarkers to identify the target condition, one or more of the patterns having similarity values corresponding to the first physiological signals of the first group or the second physiological signals of the second group that differentiate the first physiological signals of the first group and the second physiological signals of the second group.

2. The computer implemented method of claim 1,
wherein identifying at least a frequency sub-band comprises identifying a plurality of frequency sub-bands for each of the first group and the second group, the plurality of frequency sub bands corresponding to frequencies that show a higher group synchronization than other frequencies;
wherein obtaining, for each of the at least one frequency sub band, at least a corresponding pattern comprises obtaining, for each of the plurality of frequency sub bands, a corresponding time series pattern; and
wherein selecting, as functional biomarkers to identify the target condition, comprises selecting, as functional biomarkers to identify the target condition, one or more of the time series patterns having similarity values corresponding to the first physiological signals of the first group or the second physiological signals of the second group that differentiate the first physiological signals of the first group and the second physiological signals of the second group.

3. The computer implemented method of claim 1, wherein the stimulation procedure is a periodical task having a task frequency, and the frequency sub-band comprises the task frequency.

4. The computer implemented method of claim 1, wherein the first physiological signals and the second physiological signals are one of a near-infrared spectroscopy (NIRS) signal, a functional magnetic resonance imaging-blood-oxygen-level-dependent (fMRI-BOLD) signal, an electroencephalography (EEG) signal, or an hemodynamic signal, wherein the hemodynamic signal is a blood pressure or flow signal.

5. The computer implemented method of claim 4, wherein the physiological signal, obtained in a same region of the brain, in response to relative or absolute changes in a concentration of a hemoglobin chromophore.

6. The computer implemented method of claim 2, wherein the identifying the frequency sub-band comprises:
obtaining a time-frequency transform of each of the first physiological signals and the second physiological signals;
averaging the time-frequency transforms;
extracting local-maxima of the averaged time-frequency transforms; and
identifying the plurality of frequency sub-bands for the first group and the second group as frequency ranges around the local-maxima.

7. The computer implemented method of claim 6, wherein the time-frequency transform is a continuous wavelet transform.

8. The computer implemented method of claim 6, wherein the obtaining the plurality of the frequency sub-bands comprises:
computing an inverse time-frequency transform from the calculated time-frequency transform of the physiological signals for each of the frequency sub-bands to obtain a corresponding time-series for each of the first physiological signals of the first group and the second physiological signals and the second group; and
generating the time-series pattern, for each of the first group and the second group, by averaging the obtained time-series for each of the first physiological signals of the first group and the second physiological signals and the second group.

9. The computer implemented method of claim 2, wherein the selecting the one or more time-series patterns comprises:
calculating time-series similarity values between each candidate time-series pattern and the time-series of each of the first physiological signals of the first group and the second physiological signals and the second group, and
selecting, as functional biomarkers, the time-series patterns with similarity values that differentiate the groups greater than a threshold amount.

10. The computer implemented method of claim 2, wherein the identifying the frequency sub-bands comprises calculating an inter-subject synchronization measure.

11. The computer implemented method of claim 10, wherein the ISS measure comprises a phase synchronization measure.

12. The computer implemented method of claim 10, wherein the ISS measure comprises a combination of a phase and magnitude synchronization measure.

13. The computer implemented method of claim 2, wherein the one or more time-series patterns are selected based on statistical contrast at a predefined significance level.

14. The computer implemented method of claim 1, further comprising:
identifying the target condition in a subject based on the one or more time-series patterns.

15. The computer implemented method of claim 1, wherein the target condition is one of: a target mental condition, target brain disorder condition, target body physiological condition, or a combination of one or more of the target mental condition, the target brain disorder condition, and the target body physiological condition.

16. The computer implemented method of claim 1, wherein the target condition is one of: attention-deficit/hyperactivity disorder (ADHD), depression or anxiety.

17. The computer implemented method of claim 1, further comprises:
training a classifier for identifying the target condition using the similarity values.

18. The computer implemented method of claim 17, further comprises:
obtaining a physiological signal from a target subject during a stimulation; and
processing the physiological signal of the target subject in the classifier to identify whether the target condition is present in the target subject.

19. A diagnostic method comprising: identifying the target condition in a target subject by using the computer implemented method of claim 1.

20. A non-transitory computer readable medium having stored thereon a computer program including instructions that when executed cause a machine to perform a method comprising:
obtaining, based on a stimulation procedure, a first group of first physiological signals from one or more first subjects representing a target condition, and a second group of second physiological signals from one or more second subjects representing a control condition not presenting the target condition;
identifying at least one frequency sub-band for each of the first group and the second group;
obtaining, for each of the at least one frequency sub-band, at least a corresponding pattern; and
selecting, as functional biomarkers to identify the target condition, one or more patterns having similarity values corresponding to the first physiological signals of the first group or the second physiological signals of the second group that differentiate the first physiological signals of the first group and the second physiological signals of the second group.

21. An electronic device comprising:
a memory storing one or more instructions, and
a processor configured to execute the one or more instructions to:
obtain, based on a stimulation procedure, a first group of first physiological signals from one or more first subjects representing a target condition, and a second group of second physiological signals from one or more second subjects representing a control condition not presenting the target condition,
identify a plurality of frequency sub-bands for each of the first group and the second group,
obtain, for each of the at least one frequency sub-band, at least a corresponding pattern, and
select, as functional biomarkers to identify the target condition, one or more patterns having similarity values corresponding to the first physiological signals of the first group or the second physiological signals of the second group that differentiate the first physiological signals of the first group and the second physiological signals of the second group.
